(19) United States
(12) Patent Application Publication
(10) Pub. No.: US 2005/0227297 A1
(43) Pub. Date: Oct. 13, 2005

(54) Method for immunohistochemical detection of collagen in a tissue sample

(76) Inventor: Michael R. D'Andrea, Cherry Hill, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/100,656

(22) Filed: Apr. 7, 2005

Related U.S. Application Data
(60) Provisional application No. 60/560,456, filed on Apr. 8, 2004.

(51) Int. Cl. G01N 33/53; G01N 33/567; G01N 33/554; G01N 33/569

(52) U.S. Cl. 435/7.2; 435/7.32

(57) ABSTRACT

Pretreating a tissue sample with an effective amount of collagenase results in increased immunodetection of collagen and enhanced preservation of tissue morphology. Such an improved method for immunodetection of collagen can be used in immunohistochemical studies of formalin-fixed, paraffin-embedded tissue sections.

### CROSS-REFERENCE TO RELATED APPLICATIONS

This application claims priority to U.S. Application Ser. No. 60/560,456, filed Apr. 8, 2004, the entire contents of which are incorporated by reference herein.

### FIELD OF THE INVENTION

The present invention relates to methods of histochemical detection. In particular, the present invention relates to an improved method for immunohistochemical detection of a collagen in a tissue sample.

### BACKGROUND OF THE INVENTION

Collagens, a family of extracellular matrix (ECM) proteins that play a dominant role in maintaining the structural integrity of various tissues and organs, are abundant in the human body. They are found in essentially all tissues and are particularly rich in tissues such as bone, skin, tendon, cartilage, ligaments and vascular walls.

Histochemical detection of collagen plays a key role for diagnostic pathology. In many disease states, the cells either produce altered (increases or decreased) amount of collagens, or produce structurally defective collagens (Kivirikko, 1993, Ann. Med. 25: 113-126). For example, the excessive accumulation of collagen in the interstitium plays a central role in liver disease leading to fibrosis. In addition, an increased size of endoneurial collagen fibril and increased deposition of extracellular matrix has been observed in the diabetic peripheral nerve. The increased deposition of extracellular matrix is characterized by thickening of basement membrane (BM), and Type IV collagen is the key constituent of BM. Furthermore, BM in malignant and invasive epithelial tumors is either completely absent or thin and discontinuous, whereas many benign epithelial tumors have an intact BM. (Albrechtsen et al., 1981, Cancer Res. 41:5076-5081).

Immunohistochemical detection of specific collagens within tissues has been a powerful tool in investigations of morphogenesis, cell differentiation, and regeneration. The immunohistochemical detection of collagen, like most immunocytochemical staining, works the best with fresh or freshly-frozen tissue. But, because fresh or freshly-frozen tissues are not conveniently available, and sometimes frozen tissues can be unsuitable due to the poor histochemical preservation, immunohistochemical detection of collagen is commonly performed with tissues that have been preserved by fixation followed by dehydration and embedding. Preservation of tissue by fixation in a formalin solution, followed by dehydration and paraffin-wax embedding, remains the predominant method of preparation for microscopic analysis of morphology. Whilst this preservation technique may be optimal for morphological assessment, this technique has major disadvantages for subsequent immunohistochemical study as a result of the structural alteration of antigens that occurs during the processing procedure. Marked loss and often complete abolishment of immunohistochemical detection of collagens has been observed with the preserved tissue.

A range of antigen unmasking procedures has been developed to retrieve the immunoreactivities of antigens after routine tissue preparation (MacIntyre, 2001, Br. J. Biomed. Sci. 58:190-196). Two of the most popular unmasking techniques are enzyme digestion and heat-induced epitope retrieval. Pre-treatment of the formalin-fixed, paraffin-embedded tissues with wide-spectrum protease, also called general protease, has been shown to enhance immunohistochemical detection of collagens (Barsky, et al., 1984, Am. J. Clinical. Pathology 82: 191-194; Lowry et al., 1997, J. Anat., 191:376-374). Unfortunately, these wide-spectrum proteases, such as pepsin, pronase, trypsin and protease K, can digest many tissue proteins leaving the tissue morphology blurry and diffuse, and at times, make key cellular structures barely recognizable. It can be difficult to balance conditions, such as type and concentration of protease used, incubation time and temperature, to digest some but not all proteins in a tissue section while optimizing collagen detection. These conditions are often specific to the tissue types as well as fixation types. For example, structures in tissues such as brain are less subject to the side effects of the wide-spectrum protease (Example 2), but most structures (epithelium, spermatids, stroma, etc.) in other tissues with complicated histology such as kidney, gut, spleen, testis and others, are affected (Example 3). Although heat pretreatment technique has been widely used to enhance antigen detection in tissue sections (MacIntyre, 2001, supra), as shown in this invention, heat pretreatment only marginally increases immunohistochemical detection of collagens in formalin-fixed, paraffin-embedded tissues (Example 2).

There is a need to develop a new histochemical detection method that preserves the integrity of the surrounding tissue morphology while increases the immunohistochemical detection of collagens in a tissue sample.

### SUMMARY OF THE INVENTION

It has now been discovered that superior results of preserving the integrity of the surrounding tissue morphology while increasing the collagen detection are obtained when formalin-fixed, paraffin-embedded tissues are predigested with collagenase, a specific proteolytic enzyme capable of breaking native collagen.

In one general aspect, the invention therefore relates to an immunohistochemical method of detecting a collagen in a tissue sample, comprising the steps of:
a. incubating the tissue sample with a buffer solution comprising an effective amount of a collagenase and a cation required for the enzymatic activity of the collagenase;
b. exposing the tissue sample to an antibody capable of binding specifically to the collagen within the tissue sample; and
c. detecting the antibody bound to the tissue sample.

In a preferred embodiment, the tissue sample is a formalin-fixed and paraffin-embedded tissue section.

### BRIEF DESCRIPTION OF THE DRAWINGS

FIG. 1 shows the effects of various pretreatments on collagen IV immunodetection in formalin-fixed and paraffin-embedded tissues of normal human brain: (A) without using a pretreatment method; (B) using the heat pretreatment method; (C) using the pepsin pretreatment method; and (D) using the collagenase (clostridiopeptidase A type IV) pretreatment method. Note the varying intensities of collagen IV immunodetection (arrowheads) among the various pretreatment conditions. Bar=100 μm.

FIG. 2. shows improved immunodetection of collagen IV and enhanced preservation of tissue morphology by collagenase pretreatment in tissues with complicated histology. Formalin-fixed, paraffin-embedded tissues of normal human testis (A, C, E) and kidney (B, D, F) were either without any enzymatic pretreatment (A, B), or predigested with pepsin (C, D) or clostridiopeptidase A type IV (E, F). Note the varying intensities of collagen IV immuno-labelings (arrowheads) among the various pretreatment conditions. Also note the poor morphology of some of the surrounding tissue structures such as the spermatids (arrows, C) and the collecting tubule epithelium (arrows, D) with the pepsin pretreatment, as compared with that without a enzymatic pretreatment (arrows, A, B) or with the collagenase pretreatment (arrows, E, F). Bar=100 μm.

FIG. 3. shows improved immunodetection of collagen IV by pretreatment of tissues with various types of clostridiopeptidase A. Formalin-fixed, paraffin-embedded normal human spleen (A, C, E) and testis (B, D, F) tissues were pre-digested with clostridiopeptidase A type I (A, B), clostridiopeptidase A type IV (C, D), and clostridiopeptidase A type XI (E, F). Note the similar intensities of collagen IV immuno-labelings (arrowheads) among the various collagenase pretreatments and the enhanced preservation of the tissue morphology (arrows). Bar=100 μm.

FIG. 4. shows improved immunodetection of various collagens by pretreatment of tissues with a collagenase. Formalin-fixed, paraffin-embedded normal human tissues of spleen were pre-digested with clostridiopeptidase A type IV: A. immunodetection of type I collagen; B. immunodetection of type V collagen; C. immunodetection of type VI collagen; and D. immunodetection of type I, II and III collagens using a pan-collagen antibody. Bar=50 μm.

### DETAILED DESCRIPTION OF THE INVENTION AND ITS PREFERRED EMBODIMENTS

All publications cited herein are hereby incorporated by reference. Unless defined otherwise, all technical and scientific terms used herein have the same meaning as commonly understood to one of ordinary skill in the art to which this invention pertains.

The terms “including,” “comprising”, “containing”, and “having” are used herein in their open, non-limiting sense.

An “antibody” as used herein refers to an immunoglobulin molecule or immunologically active portions of an immunoglobulin molecule that has a specific amino acid sequence and binds only to an antigen or a group of antigens that are closely related. Examples of “antibodies” include IgG, IgM, IgA, IgD and IgE. Examples of immunologically active portions of immunoglobulin molecules include Fab and F(ab)′₂ fragments which can be generated by treating the antibody with an enzyme such as pepsin. Particularly, the “antibody” as used herein binds to one or more types of collagens but does not bind substantially to any other proteins within an assay sample.

An “antibody” can be a monoclonal antibody or a polyclonal antibody. The term “monoclonal antibody” or “monoclonal antibody composition” refers to a population of antibody molecules that contain only one species of an antigen binding site and are capable of immunoreacting with a particular epitope. The term “polyclonal antibody” refers to a population of antibody molecules that contain more than one species of an antigen binding sites and are capable of immunoreacting with more than one epitopes on the polypeptide. Examples of polyclonal antibody preparations are ones that contain antibodies directed against multiple epitopes on one type of collagen, but not the other types of collagens.

An “antigen” as used herein refers to a molecule containing one or more epitopes that will stimulate a host's immune system to make a humoral and/or cellular antigen-specific response. The term “antigen” is used herein interchangeably with “immunogen.” The term “epitope” as used herein refers to the site on an antigen or hapten to which a specific antibody molecule binds. The term “epitope” is used herein interchangeably with “antigenic determinant” or “antigenic determinant site”.

A “collagen” as used herein refers to a member of a family of closely related but distinct extracellular matrix proteins that play a dominant role in maintaining the structural integrity of various tissues and organs. A “collagen” can be any of the 21 known types of collagens that are encoded by more than 30 distinct genes. A “collagen” can also be any of the yet to be identified new types of collagens. Examples of “collagen” are listed in Table 1 (Kivirikko, 1993, supra; Buckwalter et al., 1995, Spine, 20:1307-1314).

TABLE 1
Examples of collagens and their occurrence

| Type | Occurrence |
|---|---|
| I | Ubiquitous |
| II | Cartilage, vitreous humour |
| III | Ubiquitous |
| IV | Basement membranes |
| V | Interstitial tissues |
| VI | Soft tissues |
| VII | Anchoring fibrils |
| VIII | Endothelium, mesenchyme |
| IX | Cartilage, vitreous humour |
| X | Hypertrophic cartilage |
| XI | Cartilage, vitreous humour |
| XII | Many |
| XIII | Many tissues |
| XIV | Skin, tendon |
| XV | Many tissues |
| XVI | Fibroblasts, keratinocytes |
| XVII | Skin hemidesmosomes |
| XVIII | Liver, kidney, placenta |
| lX | nucleus, anulus, and endplates |

A “collagenase” as used herein refers to a proteolytic enzyme capable of enzymatically cleaving collagen. Once the initial cleavage of collagen is made by a collagenase, less specific proteases complete the degradation of the collagen. Collagenases are metallo enzymes that require metal ions, such as zinc or calcium ion, for their proteolytic activities. A “collagenase” can be a “bacterial collagenase” that is found naturally associated with a bacterial cell, for example a cell of Clostridium histolyticum. Examples of “bacterial collagenases” include clostridiopeptidase A. Various types of clostridiopeptidase A as defined by the purification protocols are commercially available from Sigma (St. Louis. Mo.). A “collagenase” can also be a “mammalian collagenase” that is found naturally associated with a mammalian cell, such as a connective tissue cell. As used herein, “mammalian collagenases” include matrix metalloproteinases (MMPs), which are secreted and membrane-bound zinc-endopeptidases. Examples of MMPs are, interstitial collagenase also called MMP-1 that degrades type III collagen more efficiently than type I or type II collagen, neutrophil collagenase also called MMP-8 that is more potent in degrading type I collagen than type II or type III collagen, and collagenase 3 also called MMP-13 that has the highest affinity for Type II collagen. As used herein, “mammalian collagenases” also include the gelatinases, also known as type IV collagenases, which degrade gelatin (denatured collagen), and collagens types IV, V, VII, IX and X. Examples of gelatinases include gelatinase A (MMP-2) and gelatinase B (MMP-9), which are thought to have similar substrate specificity with respect to their substrates, and to be mostly responsible for the degradation of the collagen IV component in basement membranes. (Duffy et al., 2000, Breast Cancer Res. 2 (4): 252-257).

An “immunohistochemistry assay” or “immunostaining assay” is a biological assay that studies the biochemical composition of tissues or cells by means of detecting a specific labeling that correlates to a particular immunoreactive substance of the tissues or cells using antibodies specifically binding to the immunoreactive substance. The antibodies have the property of being capable of binding to the immunoreactive substance in highly specific combinations. The binding is characterized by its high degree of specificity and low dissociation constant. The immunoreactive substance can be any biological material that can serve as an antigen and elicit an immune response. Examples of the immunoreactive substances useful for collagen imunohistochemical assay include all types of collagens present in a tissue sample, such as Type I, II, and III collagens in connective tissues, and Type IV collagen in basement membranes.

The term “tissue sample” refers to a sample obtained from an organism (e.g., patient) or from components (e.g., cells) of an organism. The sample may be of any biological tissue. The sample may be a “clinical sample” which is a sample derived from a patient, therefore a “patient sample”, such as a biopsy. The “tissue sample” as used herein may be sections of tissues that are either fresh, or frozen, or fixed and embedded. Examples of tissue samples include, but are not limited to, tissue sections of brain, adrenal glands, colon, small intestines, stomach, heart, liver, skin, kidney, lung, pancreas, testis, ovary, prostate, uterus, thyroid and spleen, taken from a mammal, such as a human, mouse, rat, pig, dog, etc.

The term “labeled”, with regard to a labeled antibody used in an immunohistochemistry assay is intended to encompass direct labeling of the antibody by coupling (i.e., physically linking) a detectable substance to the antibody, as well as indirect labeling of the antibody so that the antibody can be detected by one or more other reagents that are directly labeled. Labels that can find use for direct labeling in the present invention include: fluorescent labels, or radioactive isotopes such as ³⁵S, ³²P, 3H, and the like. Examples of indirect labeling include detection of a primary antibody using a fluorescently labeled secondary antibody, and the like.

The term “fixation” or “fixing” of a tissue sample refers to the technique of using chemical agents called “biological fixatives” in the preparation of tissue samples, such as cytologic, histologic, or pathologic specimens, for the purpose of maintaining the existing form and structure of the constituent elements of the tissue sample. The fixation process usually involves denaturation, precipitation, or cross-linking of the constituent elements of the tissue sample with the biological fixatives.

The term “embedding” of a fixed tissue sample refers to the procedure that the sample is embedded in wax or plastic in order to prepare tissue sections for microscopical examination. The embedding medium, such as celloidin or paraffin, provides mechanical support to the tissue sample.

The present invention demonstrated that pre-incubating a tissue sample with collagenase(s) enhances collagen immunodetection without affecting non-collagen proteins thereby preserving tissue morphology. Collagenase, which is typically used to disassociate cells, has never been successfully used to enhance immunodetection of collagen in formalin-fixed, paraffin-embedded tissue sections. This new utility of collagenase for improved immunohistochemical detection of collagen is easy to use, has versatility among all types of collagens tested, for example, types I, IV, V, and VI. The present invention therefore provides a new method for immunohistochemical detection of collagen. Such a method preserves tissue structure providing accurate histological information while increases the immunohistochemical detection of collagens in a tissue sample.

In one general aspect, the invention therefore relates to an immunohistochemical method of detecting collagen in a tissue sample comprising the steps of:
a. incubating the tissue sample with a buffer solution comprising an effective amount of a collagenase and a cation required for the enzymatic activity of the collagenase;
b. exposing the tissue sample to an antibody capable of binding specifically to the collagen within the tissue sample; and
c. detecting the antibody bound to the tissue sample.

In one embodiment, the tissue sample is fresh or freshly-frozen. Methods are known to those skilled in the art to obtain a fresh or freshly-frozen tissue sample, for example, by rapid freezing followed by either freeze substitution or cryosectioning.

In a preferred embodiment, the tissue sample is preserved by fixation in a chemical solution, followed by dehydration and embedding. Methods of tissue fixation and embedding are known to those skilled in the art (for example, see review Oliver et al., 1999, Methods Mol Biol, 115:319-26). The tissue sample can be fixed in a biological fixative, such as acetone, alcohol, formalin, or paraformaldehyde. Preferably, the tissue sample is fixed in formalin. Formalin is an aldehyde-based fixative produced when formaldehyde gas is dissolved in aqueous solution. For example, a fixative containing 0.5% glutaraldehyde and 2% formaldehyde generally is suitable for a wide variety of tissue fixation. Fixation of the tissue sample can be achieved by immersing the sample in the fixative for a period of time, such as 30 min to 1 hour. Alternatively, fixation of the tissue sample can be achieved by the combination of the chemical fixative and the heating action, such as microwaving. Parameters include, but are not limited to the type of tissue, composition of the fixative, time and temperature of fixation, can affect the ability to immunolabel a particular antigen. Optimal fixation conditions suitable for collagen immunochemical detection can be determined experimentally by varying these parameters.

An exemplary method of fixing a tissue sample comprises the steps of: 1) rinsing the tissue sample in a buffer (such as phosphate buffer saline) and incubating the sample in a fixative (such as 0.5% glutaraldehyde and 2% formaldehyde, or 95% alcohol), for a period of time (such as 30 min to 1 h or days); 2) rinsing the sample in the buffer again; 3) quenching free aldehyde groups (such as by rinsing the sample in 0.1% glycine); and optionally 4) fixing the sample in a second fixative (such as 2% OsO4 in 0.1M cacodlylate buffer) for a period of time (such as 1 h), and then rinsing the sample again (such as in 0.1M cacodlylate buffer).

Following fixation, the tissue sample is routinely dehydrated with increasing concentrations, up to absolute, of organic solvents, such as ethanol, methanol, or acetone. The dehydrated tissue sample is impregnated with paraffin-wax for a period of time, and then embedded in fresh wax in a embedding mold. The methods of dehydrating and embedding a fixed tissue sample are known to those skilled in the art. Tissue sections can be cut, for example using a microtome, from the fixed and embedded tissue for microscopic analysis.

Tissue samples that are fixed and embedded can also be obtained from commercial sources, such as the human checkerboard tissue blocks from Dako (Carpenturia, Calif., Cat. No: T1068) or Biomeda (Foster City, Calif., Cat. No: M89).

According to the method of this invention, prior to immunohistochemical detection of a collagen, the tissue sample is incubated with an enzyme solution comprising an effective amount of a collagenase and an ion required for the enzymatic activity of the collagenase. The “effective amount of a collagenase” as used herein, refers to the amount of a collagenase that when incubated with a tissue sample prior to collagen detection is capable of increasing the immunohistochemical detection of collagen in the sample while preserving tissue structure for accurate histological information about the sample. Parameters such as the type of tissue studied, techniques used for fixation and embedding of the tissue, the type of collagenase used, the type of collagen to be detected, time and temperature of incubation, etc., can affect the effective amount of a collagenase. The effective amount of a collagenase as well as other parameters for the assay, can be determined experimentally.

Any type of collagenases, including bacterial collagenases or mammalian collagenases, can be used in the method of the invention. Pretreatment of the tissue sample with bacterial collagenases obtained from Sigma such as clostridiopeptidase A type I, IV, and XI, all enhanced the immunohistochemical detection of collagen type IV as described in Example 4.

In one embodiment of the invention, the effective amount of a collagenase is the amount of collagenase in an enzyme solution with collagenase concentrations ranging from 10 μg/ml to 10 mg/ml, and the volume of the enzyme solution is sufficient to cover said tissue sample. Preferably, the effective amount of a collagenase is the amount of the collagenase in an enzyme solution with collagenase concentration of 1 mg/ml, and the volume of the enzyme solution is sufficient to cover said tissue sample. For example, the effective amount of collagenase for a formalin-fixed and paraffin-embedded tissue section on a microscopic slide is about 2-3 drops of collagenase solution with enzyme concentrations ranging from 10 μg/ml to 10 mg/ml, preferably at about 1 mg/ml.

In a preferred embodiment of the invention, the tissue sample is incubated with an enzyme solution comprising an effective amount of a collagenase and an ion required for the collagenase activity at a temperature ranging from 37 to 45° C. Most preferably, the incubation temperature is about 40° C. It was reported previously that pretreatment of formalin-fixed and paraffin-embedded tissues with bacterial collagenase or Type IV collagenase at a temperature of 37° C. for varying time courses ranging from 30 minutes to 24 hours, was ineffective in enhancing immunohistochemical detection of collagens associated with basement membranes (Barsky, 1984, Am. J. Clin. Pathol. 82:191-194). However, it was observed in this invention that pretreatment of formalin-fixed and paraffin-embedded tissues with collagenase at a temperature of 37° C. resulted in some enhancement of collagen immunohistochemical detection, although not as much as when the incubation was performed at 40° C. The discrepancy of these results is possibly due to the lack of calcium, an ion that is required for the collagnase activity, in the incubation buffer of the previous study. Accordingly, in the method of the present invention, an ion required for the enzymatic activity of the collagenase, such as the calcium or zinc ion, is included in the enzyme solution. The preferred enzyme solution includes calcium ion, Ca²⁺. Zinc ion, Zn²⁺, is required for collagenase activity, but it is tightly bound to the collagenase during purification. Additional Zn²⁺ should not be necessary as long as no chelator is added to the enzyme solution.

In yet another embodiment of the invention, the tissue sample is incubated with an enzyme solution comprising an effective amount of a collagenase and a cation required for the enzymatic activity of the collagenase for a time period ranging from 10 minutes to 24 hours, preferably for a time period ranging from about 1 to 4 hours, and most preferably for a time period of 1 hour.

According to the method of this invention, after the tissue sample is pre-digested with an effective amount of a collagenase, the collagen in the pretreated tissue sample can be detected by an immunohistochemical detection method comprising the steps of exposing the tissue sample with an antibody capable of binding specifically to the collagen within the tissue sample, and detecting the antibody bound to the tissue sample.

Antibodies that are useful to this invention can be monoclonal or polyclonal antibodies that bind specifically to any type of collagens. The antibodies can be derived from a variety of sources, including but not limited to, goat, mouse, rat, sheep, horse, chicken, and rabbit. Methods are known to those skilled in the art to produce an antibody that binds specifically to a collagen. For example, polyclonal antibodies can be raised by immunizing suitable subject animals such as mice, rats, guinea pigs, rabbits, goats, horses and the like, with rabbits being preferred, with a collagen with or without an immune adjuvant. Monoclonal antibodies (mAb) can be produced by growing hydridoma cells in tissue culture media, wherein the hydridoma cells can be obtained by mixing the splenic lymphocytes from a collagen immunized inbred mice, preferably Balb/c, with an appropriate fusion partner, preferably myeloma cells, under conditions that will allow the formation of stable hybridomas. Antibodies that are useful to this invention can also be obtained from commercial sources, for example, rabbit polyclonal pan-collagen antibodies (Chemicon, Temecula, Calif.) that bind to a collection of several types of collagens such as Type I, II, and III collagens, goat polyclonal collagen I antibody (Santa Cruz, Santa Cruz, Calif.) that binds specifically to Type I collagen, mouse monoclonal collagen IV antibody (Dako) that binds specifically to Type IV collagen, goat polyclonal collagen V antibody (Santa Cruz) that binds specifically to Type V collagen, and goat polyclonal collagen VI antibody (Santa Cruz) that binds specifically to Type VI collagen.

According to the invention, an antibody that binds specifically to a collagen is incubated with the collagenase pre-digested tissue sample in an aqueous solution to allow specific binding of the antibody to the collagen within the sample. The binding of the antibody to the collagen can be detected by a variety of methods known to those skilled in the art (Mokry, 1996, Acta Medica (Hradec Kralove), 39:129-40). The binding can be detected by a direct method using a labeled antibody, for example a fluorescently labeled antibody that binds specifically to a collagen, and detecting the labeling.

Preferably, the binding of the collagen specific antibody in the tissue sample can be detected by an indirect method, for example, by an enzyme/anti-enzyme complex method such as horseradish peroxidase/anti-peroxidase and alkaline phosphatase/antialkaline phosphatase methods. Another example of the indirect detection method is based on avidin-biotin interactions, such as the methods of bridged avidin-biotin, avidin-biotin complex, and labeled avidin-biotin. Other examples of indirect detection methods include methods based on protein A-antibody interaction, and hapten antibody anti-hapten methods.

The detection step is usually performed by chromogenic detection. For example, a secondary antibody labeled with an enzyme such as horseradish peroxidase or alkaline phosphatase, is first incubated with the tissue sample under condition to allow specific binding of the secondary antibody to the antibody that binds specifically to collagens (primary antibody) in the sample. The unbound secondary antibody is then washed off, and the amount of the secondary antibody that remains with the sample is detected using an enzyme substrate such as 3,3′-diaminobenzidine or nitroblue tetrazolium chloride/5-bromo-4-chloro-3-indolyl-phosphate (toluidine salt) respectively. The enzyme attached to the secondary antibody is capable of converting the substrate into a colored precipitate that is visible under light microscopy.

### EXAMPLE 1

### Methods and Materials

Human checkerboard tissue blocks (Dako, Carpenturia, Calif.; Biomeda, Foster City, Calif.) that are formalin-fixed, paraffin-embedded were routinely processed for immunohistochemistry (D'Andrea et al., 2003; Neuroscience Letters 333(3): 163-166). The tissues assayed in this studies were brain (n=10), adrenal glands (n=10), colon (n=6), small intestines (n=2), stomach (n=2), heart (n=6), liver (n-10), skin (n=3), kidney (n=8), lung (n=10), pancreas (n=10), testis (n=8), ovary (n=8), prostate (n=8), uterus (n=8), thyroid (n=10) and spleen (n=10). Tissue sections on microscopic slides were dewaxed and re-hydrated prior to use according to routine methods (D'Andrea et al., 2003, supra). The individual collagenases used in this study were bacterial collagenase, the clostridiopeptidase A types IA, IV, and XI (Sigma, St. Louis. MO, Product number, C0130, C5138, and C7657), which were prepared (1 mg/ml) in collagenase buffer (containing 11.47 g/l of TES free acid (Sigma, Product No T-1375), 0.053 g/l of calcium chloride, dihydrate (Sigma, Product No C-3881) in deionized H₂O, the pH of the buffer was adjusted to 7.4 with 1M NaOH). The proteases used in this study were pepsin solution (Invitrogen Corp., Carlsbad, Calif., Catalog No: 750102), trypsin (1 mg/ml; Dako, Carpenturia, Calif.) and protease K (1 mg/ml, Dako, Carpenturia, Calif.).

Before the immunohistochemical assay, tissue sections on microscopic slides were grouped according to no pretreatment, enzymatic pretreatment or heat pretreatment. For collagenase pretreatment, collagenases (10 μg/ml-10 mg/ml, routinely about 1 mg/ml) in collagenase buffer were preheated at 37-45° C. (routinely about 40° C.) for 5 min. Then, about 2-3 drops of the enzyme solution were placed on each microscopic slide to cover the tissue section. Coverslips were gently placed on top of the tissue sections on the slides, and the slides were incubated in a moist chamber (slide moat, Boekel Scientific, Feasterville, Pa., Model 240000) for 10 min to overnight (routinely about 1 h) at 37-45° C. (routinely about 40° C.). For general protease pretreatment, pepsin, trypsin, or protease K enzyme solution as described above was preheated in a 37° C. water bath for 5 minutes. Then, about 2-3 drops of the enzyme solution were placed on each microscopic slide to cover the tissue section. Coverslips were gently placed on top of the tissue sections on the slides, and the slides were incubated in a moist chamber for about 10 min at 37° C. For heat pretreatment, tissue sections on the microscopic slides were microwaved (Energy Beam Sciences, Inc., MA) (45°W, 98° C. for 2 times 3 min) in Target buffer (Dako, Carpenturia, Calif.), cooled, placed in phosphate-buffered saline (pH 7.4, PBS) and treated with 3.0% H₂O₂ for 10 min at room temperature. For heating accuracy, the same number of slides (n=24) placed in a microwavable slide rack was always heated together regardless of the number of slides with tissues on them (D'Andrea et al., 2003, supra).

For the immunohistochemical assay on collagens, all incubations (30 min each) and washes were performed at room temperature. Normal blocking serum (Vector Labs, Burlingame, Calif.) was placed on all tissue slides for 10 min. After a brief rinse in PBS, sections were incubated with the primary antibodies choosing from rabbit polyclonal pan-collagen (1:2,000; Chemicon, Temecula, Calif., Cat No: MAB1334), goat polyclonal collagen I (1:1,500; Santa Cruz, Santa Cruz, Calif., Cat No: SC-8784), mouse monoclonal collagen IV (1:200; Dako, M785), goat polyclonal collagen V (1:150, Santa Cruz, Cat No: SC-9851) and goat polyclonal anti-collagen VI (1:25; Santa Cruz, Cat No: SC-9854). For consistency and comparative analyses, the same primary antibody titers were used throughout each group of experiments. Slides were then washed in PBS and incubated with goat anti-rabbit, rabbit anti-goat or horse anti-mouse biotinylated secondary antibodies (VECTASTAIN ABC Kit, Vector Labs, Burlingham, Calif., Cat No: PK-6105). After washing in PBS, the avidin-biotin-horseradish peroxidase complex reagent (Vector Labs) was added. All slides were washed and treated with 3,3′-diaminobenzidine (Biomeda, S10) 2 times 5 min each, rinsed in distilled water, and counterstained with hematoxylin (Sigma, St. Louis, Mo., MHS-16).

### EXAMPLE 2

### Enhancement of Immunohistochemical Detection of Collagen IV by Enzymatic Pretreatment in Formalin-fixed, Paraffin-embedded Tissues of Normal Human Brain

The effects of various pretreatment methods on immunohistochemical detection of collagen in formalin-fixed, paraffin-embedded tissues of normal human brain are compared. Similar experiments can be performed to compare the effects of pretreatment methods on other types of tissue samples, such as fresh tissue section, frozen tissue section, or other types of fixed and embedded tissues.

Checkerboard tissue blocks of human normal brain (Dako, Carpenturia, Calif.; Biomeda, Foster City, Calif.) were used for this study. The tissues were pretreated by heat, by general protease such as pepsin (prediluted from the vendor), trypsin (1 mg/ml) or protease K (1 mg/ml), or by collagenase, clostridiopeptidase A type IV (1 mg/ml), using procedure described in Example 1. The immunohistochemical assay on collagen UV was performed as described in Example 1 using mouse monoclonal collagen IV (1:200; Dako).

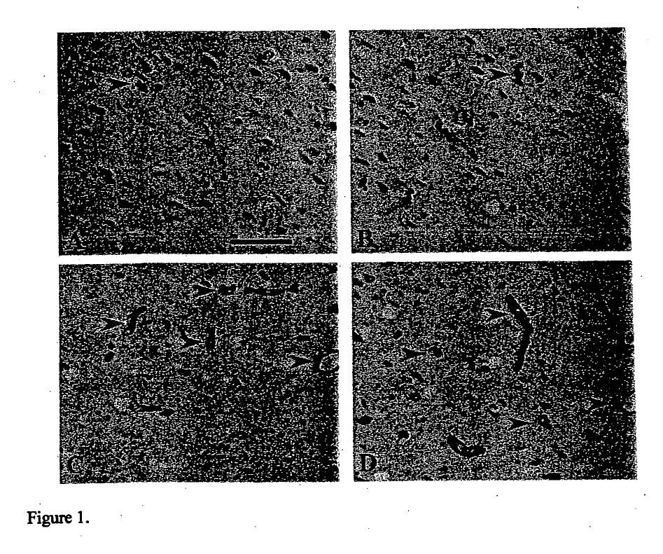

FIG. 1 shows the effects of various pretreatment conditions on collagen IV immunodetection in formalin-fixed, paraffin-embedded tissues of normal human brain. The presence of collagen immunolabeling was presented as brown staining. No observable labeling was observed in the negative control slides wherein the primary antibody, i.e., the antibody binds specifically to collagen IV, was replaced with the antibody dilution buffer (Zymed Labs, South SanFrancisco, Calif.). Barely any collagen IV immunolabeling was detected when the tissue was not pretreated (FIG. 1A). Pretreating the tissue with heat resulted in slightly more collagen IV immunodetection (arrowheads) (FIG. 1B). Dramatically increased amount of collagen IV was detected in tissues pretreated with pepsin (FIG. 1C). The enhancement of collagen immunohistochemical detection by pepsin pretreatment is consistent with results reported previously (Barsky, 1984, supra). Contrast to that of previous report (Barsky, 1984, supra), it was observed through out the invention that pretreatment with trypsin or protease K produced similar increased collagen detection as compared to the pepsin. Pretreating the tissue with collagenase, clostridiopeptidase A type IV, resulted in equal intensities and details of collagen IV immunohistochemical detection as compared to those resulting from pepsin pretreatment. (FIG. 1D). Note the fine details of the collagen labeling around the smallest capillaries (FIGS. 1C, D). Tissues that were pretreated with both heat and collagenase did not produce more enhancement of collagen immunodetection as compared to tissues that were pretreated with collagenase alone. These data indicate that the beneficial effects of enzymatic pre-treatment were greater than that of heat pretreatment at enhancing the collagen IV immunodetection in formalin-fixed, paraffin-embedded tissues of normal human brain. Increased collagen detection may not be attributed to the removal or hydration of the cross-linking formalin bonds through the heat methods, rather it may due to the digestion or removal of proteins from accessible collagen epitopes during the pan-protease pretreatment, or the digestion of the collagen to create more epitopes during the collagenase pretreatment.

### EXAMPLE 3

### Improved Detection and Preservation of Tissue Morphology in Tissues with Complicated Histology by Collagenase Pretreatment

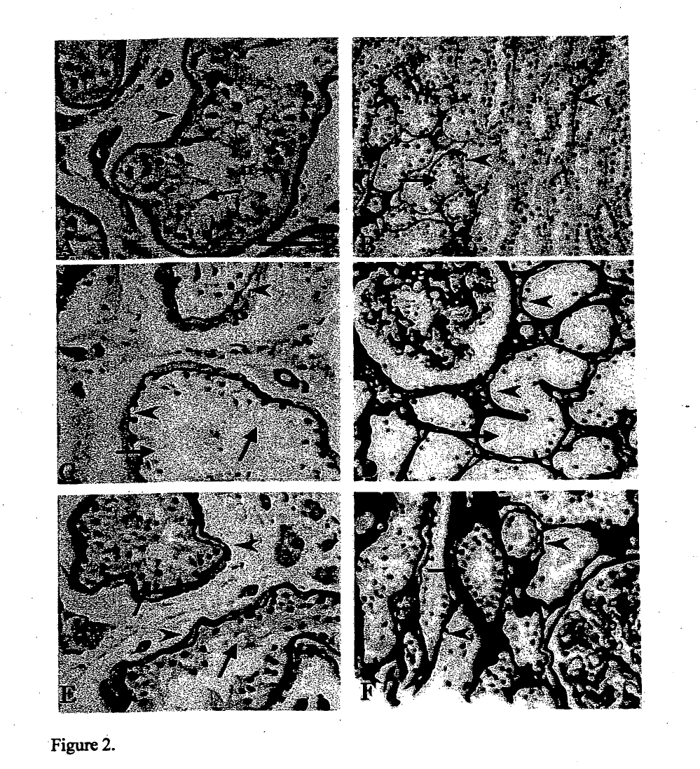

One common side effect of general protease pretreatment is that such type of pretreatment often results in dramatic changes in the morphology of the surrounding areas among tissues with complicated histology such as kidney, gut, spleen, testis and others. For example, the morphology of the spermatid and surrounding testicular structures of the testis (arrows, FIG. 2C) and the epithelium of the collecting tubules of the kidney (arrows, FIG. 2D) were almost entirely missing due to over-digestion by the pepsin, but were nicely preserved without the protease treatments (FIGS. 2A, B). Pretreatment with trypsin and protease K produced similar side effects as that of pepsin. Although the heat pretreatments preserved the tissue morphology, such type of pretreatment did not enhance the collagen immunodetection as compared to the enzyme pretreatment (Example 2). When tissues were pretreated with the collagenase, clostridiopeptidase A type IV, not only equal intensity of immunolabeling was detected as compared to tissues that were pretreated with the widely used pan-protease, but most importantly, the morphology of the surrounding tissue was preserved in the testicular spermatids (arrows, FIG. 2E) and in the collecting tubule epithelium of the kidney (arrows, FIG. 2F). Similar beneficial results, including the preservation of epithelial detail and other pertinent structures, were observed with many other tissues with complicated histology, such as the large and small intestines, spleen, stomach, and so forth, when the tissues were pretreated with collagenase.

### EXAMPLE 4

### The Versatility of Method of Collagenase Pretreatment

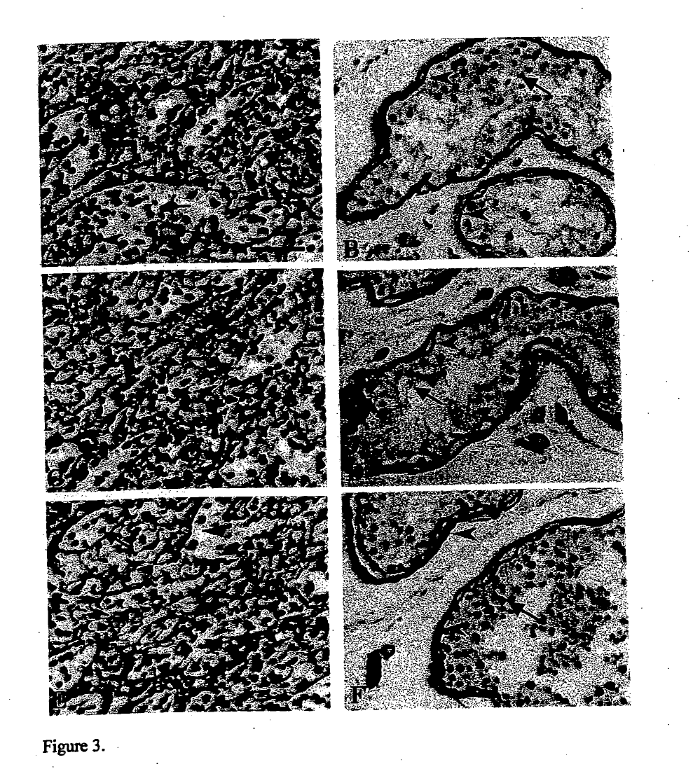

Enhancement of immunohistochemical detection of collagen type IV was also observed in tissues that were pretreated with other collagenases besides bacterial collagenase, clostridiopeptidase A type IV. For example, bacterial collagenase, clostridiopeptidase A type I, type IV, or type XI was used to pretreat representative examples of the normal human spleen and testis under identical assay conditions, 1 mg/ml of collagenase for 1 hour incubation at about 40° C. It was found that pretreatment of the tissues with clostridiopeptidase A type I, type IV or type XI resulted in similar beneficial effects at improving the collagen IV immunolabeling intensity (arrowheads) and preserving cellular structures (arrows) (FIG. 3).

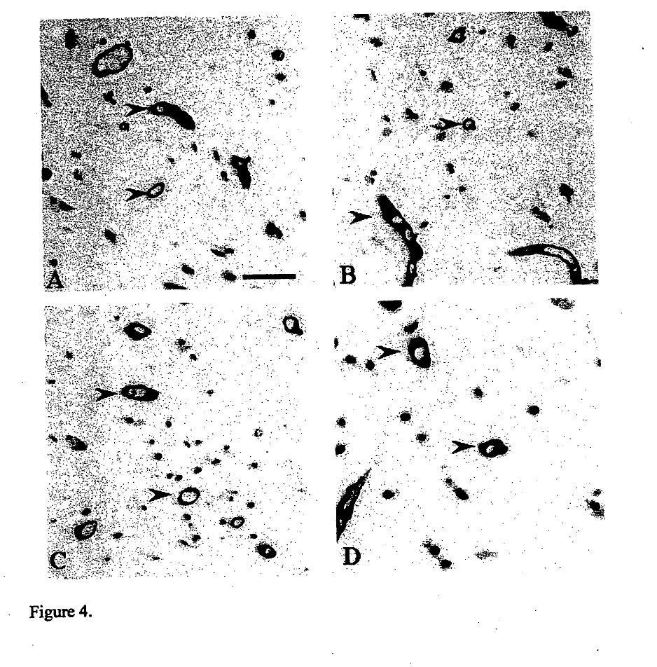

Enhancement of immunohistochemical detection of many other types of collagens besides Type IV collagen was also observed in tissues that were pretreated with clostridiopeptidase A type IV. For example, pretreatment of the normal human tissue spleen with clostridiopeptidase A type IV resulted in enhancement of immunohistochemical detection of collagen type I (arrowheads, FIG. 4A), type V (arrowheads, FIG. 4B), type VI (FIG. 4C), and all collagens using a pan-collagen antibody (FIG. 4D). The enhancement is shown as both the increased intensity of collagen immunolabeling and the well preserved tissue morphology.

Enhancement of immunohistochemical detection of collagen was observed under a variety of assay conditions. Routinely, the tissue section was predigested with an effective amount of a collagenase for 1 hour at 40° C. However, the effective amount of collagenase, the time and temperature for the pre-digestion incubation can be varied within wide ranges. For example, up to 4 hours of pre-digestion incubation produced similar results as that of 1 hour pre-digestion incubation. Also, some enhancement of collagen immunohistochemical detection was observed even when the pretreatment was performed at about 37° C. Collagenase concentrations, ranging from 10 μg/ml to 100 mg/ml, were experimented.

The versatility of the method of collagenase pretreatment provides huge benefits over the narrow operating conditions of the method of general protease pretreatment. For example, in performing the method of general protease pretreatment, digesting the tissues with just 1 to 2 additional minutes can lead to over-digestion and consequently, poor tissue morphology. Furthermore, the type of tissue fixative also affects parameters used for general protease pretreatment whereby the alcoholic fixatives require minimal digestion while the formalin fixatives require additional time. Each of the narrow operating conditions for general protease pretreatment, if not carefully examined, could produce poor morphology in spite of terrific collagen immunolabeling.

This is the first time collagenase was used successfully to pretreat formalin-fixed, paraffin-embedded tissue sections to increase collagen immunolabeling. The beneficial effects of the method of collagenase pretreatment go well beyond increasing the immunodetection of collagen to include well preservation of tissue morphology and ease of use. The method of collagenase pretreatment allows more histological information to be analyzed in the context of enhanced collagen immunolabeling. In addition, the method of collagenase pretreatment has versatility such that enhancement of immunohistochemical detection of a collagen was observed under a variety of assay conditions.

Although the various aspects of the invention have been illustrated above by reference to examples and preferred embodiments, it will be appreciated that the scope of the invention is defined not by the foregoing description, but by the following claims properly construed under principles of patent law.

1. An immunohistochemical method of detecting a collagen in a tissue sample, comprising the steps of:
a. incubating the tissue sample with a buffer solution comprising an effective amount of a collagenase and a cation required for the enzymatic activity of the collagenase;
b. exposing the tissue sample to an antibody capable of binding specifically to the collagen within the tissue sample; and
c. detecting the antibody bound to the tissue sample.

2. The method according to claim 1, wherein the tissue sample is fixed in a solution containing an aldehyde.

3. The method of claim 2, wherein the tissue sample is fixed in a solution containing formalin.

4. The method of claim 2, wherein the tissue sample is paraffin-embedded.

5. The method of claim 1, wherein the tissue sample is a tissue section.

6. The method of claim 5, wherein the tissue section is selected from the group consisting of tissue sections of brain, adrenal glands, colon, small intestines, stomach, heart, liver, skin, kidney, lung, pancreas, testis, ovary, prostate, uterus, thyroid and spleen of a mammal.

7. The method according to claim 1, wherein the collagenase is of bacterial or mammalian origin.

8. The method of claim 7, wherein the collagenase is capable of catalyzing the degradation of one or more types of collagen.

9. The method of claim 8, wherein the collagenase is capable of catalyzing the degradation of one or more types of collagen selected from the group consisting of type I, type II, type III, type IV, type V, type VI, and type XI collagen.

10. The method of claim 1, wherein the effective amount of a collagenase is about 10 μg/ml to about 10 mg/ml in the buffer solution.

11. The method of claim 10, wherein the effective amount of a collagenase is about 1 mg/ml in the buffer solution.

12. The method of claim 1, wherein the cation is a zinc or calcium cation.

13. The method of claim 1, wherein the antibody is capable of binding specifically to one or more types of collagen.

14. The method of claim 13, wherein the collagen is selected from the group consisting of type I, type II, type III, type IV, type V, and type VI collagen.

15. The method of claim 1, wherein the incubating step comprises incubating the tissue sample at a temperature ranging from about 37 to 45° C.

16. The method of claim 15, wherein the temperature is about 40° C.

17. In a method of detecting a collagen in a tissue sample, the improvement comprising the step of: incubating the tissue sample with a buffer solution comprising an effective amount of a collagenase enzyme and a cation required for the enzymatic activity of the collagenase;

18. The method according to claim 17, wherein the tissue sample is fixed in a solution containing an aldehyde.

19. The method according to claim 17, wherein the collagenase is of bacterial or mammalian origin.

20. The method of claim 19, wherein the collagenase is capable of catalyzing the degradation of one or more types of collagen.

21. The method of claim 17, wherein the cation is a zinc or calcium cation.
